(19) United States
(12) Patent Application Publication
(10) Pub. No.: US 2022/0226342 A1
(43) Pub. Date: Jul. 21, 2022

(54) COMPOSITIONS AND METHODS OF TREATING LIVER CANCERS

(71) Applicant: The Johns Hopkins University, Baltimore, MD (US)

(72) Inventors: Florin M. Selaru, Clarksville, MD (US); Ling Li, Baltimore, MD (US)

(21) Appl. No.: 17/623,060

(22) PCT Filed: Jun. 29, 2020
(86) PCT No.: PCT/US2020/040075

§ 371 Date: Dec. 27, 2021

Related U.S. Application Data
(60) Provisional application No. 62/867,991, filed on Jun. 28, 2019.

(51) Int. Cl. A61K 31/55; A61P 35/00

(52) U.S. Cl. CPC A61K 31/55; A61P 35/00

(57) ABSTRACT

Described herein are methods for treating liver tumors and cancer in a subject comprising the steps of administering a suppressor of protein translation to a subject in need thereof. Examples of liver cancer include hepatocellular carcinoma (HCC), cholangiocarcinoma, or a combination thereof. Examples of a suppressor of protein translation includes omacetaxine mepusuccinate, and the methods include combinations with other chemotherapeutic agents.

### REFERENCE TO RELATED APPLICATIONS

This application claims the benefit of U.S. Provisional Patent Application No. 62/867,991, filed on Jun. 28, 2019, which is hereby incorporated by reference for all purposes as if fully set forth herein.

### BACKGROUND OF THE INVENTION

Hepatocellular Carcinoma (HCC) is among the most frequent malignancy in adults, with approximately 765,000 new cases as well as 800,000 global deaths each year^{1, 2.} The World Health Organization (WHO) estimates that by 2030, there will be a yearly death rate of more than 1 million per year due to HCC. In the United States specifically, the death rate due to HCC has seen a sharp increase of 43% over a span of only 16 years (2000-2016)³. Furthermore, HCC has a dismal survival of only 18% at 5 years from diagnosis³. This poor survival makes HCC the second most lethal cancer³. The only curative approach to HCC is surgical—surgical resection or liver transplantation. Nonetheless, more than 70% of cases are diagnosed at advanced stages, when curative surgery is not an option any longer⁴. Moreover, approximately 70% of HCC patients suffer from recurrence and/or metastasis within 5 years of surgical intervention⁵.

For advanced HCC, first line systemic chemotherapy agents sorafenib and lenvantinib, as well as second line agents regorafenib, cabozantinib and ramucirumab achieve fairly low response rates⁶⁻⁹. In the pivotal Sorafenib Hepatocellular Carcinoma Assessment Randomized Protocol (SHARP) trial, sorafenib extended the survival of HCC patients from a median survival of 7.9 to 10.7 months⁹. In another trial, lenvatinib demonstrated non-inferior efficacy when compared to sorafenib that allowed its approval⁸. The development of effective systemic therapies against HCC has been hindered, at least in part, by the lack of accurate HCC models. Recent work demonstrated that three-dimensional (3D) human primary patient derived organoids (PDO) accurately capture original HCC tissue architecture, as well as maintain the genomic, epigenetic and molecular heterogeneity features of original tumor source even after long-term expansions¹⁰⁻¹⁵. In addition, HCC PDOs can be utilized for drug screening, as the inventors have done in previous studies^{10,13}.

Omacetaxine mepusuccinate (formerly known as homoharringtonine) a suppressor of protein translation, is approved by the Food and Drug Agency (FDA) for relapsed/refractory chronic or accelerated phase chronic myelogenous leukemia (CML), that is resistant to two or more tyrosine kinase inhibitors (TM)²⁰. Unlike TKIs, omacetaxine does not bind to BCR-ABL, and also is not affected by resistance inducing BCR-ABL mutations¹⁶⁻¹⁹. Omacetaxine acts though blocking global protein synthesis, with a disproportionate effect on those proteins, such as BCR-ABL, that have short half-lives and depend on continuous protein synthesis²⁰. The toxicity prolife for omacetaxine is favorable at the FDA approved dosage. Main side effects include hematologic depletion (thrombocytopenia, anemia, and neutropenia) and diarrhea, while infectious complications occur in approximately 5% of patients²¹. Omacetaxine has therefore proven as a vital therapeutics for CML and is currently investigated as single agent as well as part of multiple drug combinations in other CML patient cohorts²⁰. However, to date, omacetaxine is the only agent in its class that has shown any clinical activity²⁰.

### SUMMARY OF THE INVENTION

Using the HCC PDO methods, the inventors have successfully identified compounds for treating liver cancer cells or populations of cells.

In accordance with a first embodiment, the present invention provides a method for treating a solid cancer or tumor in a subject comprising the steps of administering to the subject an effective amount of a suppressor of protein translation to the subject.

In accordance with a second embodiment, the present invention provides a method for treating a liver tumor in a subject comprising the steps of administering to the subject an effective amount of a suppressor of protein translation to the subject.

In accordance with a third embodiment, the present invention provides a method for treating a liver tumor in a subject comprising the steps of administering to the subject an effective amount of omacetaxine to the subject.

In accordance with a fourth embodiment, the present invention provides a method for treating a liver tumor in a subject comprising the steps of administering to the subject an effective amount of a suppressor of protein translation and at least one additional biologically active agent to the subject.

In accordance with a fifth embodiment, the present invention provides a method for treating a liver tumor in a subject comprising the steps of administering to the subject an effective amount of a suppressor of protein translation and at least one additional chemotherapeutic agent to the subject.

### BRIEF DESCRIPTION OF THE DRAWINGS

FIG. 1A-1B. Characterization of human HCC derived Organoids compared with the original source of HCC tissue. Immunofluorescence staining of patient HCC tissue and the HCC PDOs. As showed both tissue and PDOs are positive of HCC markers AFP and Hep Par1, and both express LGR5 (stem cell marker), CK19 (hepatocyte and biliary marker), as well as epithelial marker (EPCAM).

FIG. 2A-2E. 10 μM drug screening and IC₅₀ calculation across 40 PDOs from 20 HCC patients. (2A). 10 μM drug screening of omacetaxine and sorafenib, regorafenib, lenvatinib and cabozantinib on 40 PDOs derived from 20 HCC patients. X axis—the 40 PDO lines, Y axis—Cell viability. (2B). IC₅₀ curve of 40 PDO lines, x-axis—omacetaxine dose, Y axis—Relative cell viability. (2C). Upper panel shows the bright field pictures a HCC PDO line treated with different dose of omacetaxine; lower panel shows live/dead cell staining of this same patient derived PDOs treated with different dose of omacetaxine. (2D). IC₅₀ distribution of 40 HCC PDOs. X axis—PDO lines, Y axis—IC₅₀ number of omacetaxine for each PDO. (2E) Each panel shows the microscopic image collected at 96 hours after treatment with each drug, at a concentration of 10 μM.

FIG. 3A-3H. Mechanism of Omacetaxine action on different HCC patient derived organoids lines. (3A). Flow cytometry Histograms showing the fluorescence signal of OP-Puro conjugated with Alexa Fluor 488 (OP-Puro-AF488) incorporated in the HCC26-3 PDO line after 36 h treatment with Mock (0.1% DMSO), and omacetaxine (150 nM and 700 nM) as well as CHX (100 μg/ml) treated for 30 min as positive control, n=3.* P<0.001 Not significant (NS). (3B). Flow cytometry plots showed the percent of protein synthesis rate in 0.15 μM, 0.7 μM and 0 μM (vehicle with 1% DMSO) of concentration of omacetaxine on 6 HCC PDO lines. (3C) The mean fluorescence intensity ratio reflected protein synthesis rate of the plots data in (a). (3D-3H). Flow cytometry analysis of 5 oncoproteins (c-MYC, Beta-catenin, XIAP, MET and Cyclin D1) were suppressed with different dose of omacetaxine treatment on 6 PDO lines.

FIG. 4A-4D. Proliferation assay of HCC PDOs treated with omacetaxine in vitro. Six HCC PDO lines treated with different dose of omacetaxine (0 μM, 150 nM, 0.30 and 700 nM), then cells were subjected to proliferation assay: (4A and 4C) Flow cytometery analysis of BrdU incorporated PDO lines showing proliferation of six HCC PDO lines treated with omacetaxine. Left panel shows the gating plot shows the BrdU staining positive from each drug treatment condition. Right represent the qualification data. (4B and 4D).Ki67 expression from 6 HCC PDO lines treated with omacetaxine were analyzed with flow cytometry. Left panel displayed the plot data of each PDO lines with different dose treatment. Right panel shows the quantification bar data.

FIG. 5A-5E. Apoptosis analysis of HCC PDOs treated with omacetaxine in vitro. Organoids were plate on 24 well plate, omacetaxine at 700 μM, 350 μM were added to cell culture medium for 96 h. Then cells were collected and subjected to the analysis below. (5A and 5C). Flow cytometry analysis of 4 HCC PDOs apoptosis using Annexin V/7AAD assay. Left panel shows the overlapping plots of Annexin V and 7AAD, X axis shows Annexin V stained positive cells, Y axis is 7AAD positive cells. Right panel shows the percent of early apoptosis (Annexin V⁺7AAD⁻) and late apoptosis (Annexin V⁺7AAD⁺) of each PDOs at different dose condition showed above. (5B and 5D). Flow cytometry analysis of cleaved caspase 3 expression of omacetaxine treated HCC PDOs. Left Panel shows the plots data and right panel represents the quantification bar data. Data were showed as the mean±S.E.M from 3 independent experiments. P<0.001.

FIG. 6A-6B. Cell cycle analysis of omacetaxine treated HCC PDOs. Cell cycle analysis showed that omacetaxine causes G0/S phase arrest in 6 HCC PDOs, and the arrested percent increased with the increase of drug dose. (6A) The histogram of cell cycle distributions. (6B) Bar graph of each PDO lines with different doses of omacetaxine treatment, for all 6 HCC PDOs, with the omacetaxine dosing rising, the G0/G1 increased to around 90%, which demonstrate that omacetaxine causes G0/S check in HCC PDOs.

FIG. 7A-7I. Omacetaxine reduced the tumor size and suppress metastasis and increase the survive span in HCC PDX model in vivo. (7A and 7E) Growth of 50 PDX and 26-3 PDX treated with omacetaxine, tumor size were measured at the indicated time point. (7B and 7F) display the body weight changes during the treatment period of the two PDX model. (7C and 7G) Representative images of tumor mass at Day 60 (50 PDX) and D30 (26-3 PDX), upper panel—the control group, lower panel—the omacetaxine treated group. (7D and 7H) Tumor weight at the omacetaxine treatment pointed at day 60 (50 PDX) and at day 30 (26-3 PDX) as showed 7C and 7G separately. (7I) Survival of mice bearing 26-3 PDX after treatment. Significance was determined with log rank test. (7J) HE staining of 26-3 PDX treated with omacetaxine and control. P<0.001. Error bars represent Mean±standard deviation. Not significant (NS). (7J). H&E staining of 26-3 PDX treated with omacetaxine vs control.

FIG. 8A-8G. Omacetaxine inhibits tumor cell proliferation and increases apoptosis and suppressed HCC oncoprotein expression in vivo. (8A-8D) Immunofluorescent staining of Ki67 and cleaved caspase 3 expression in 26-3 PDX model. 8A-8B show the IF staining pictures, 8C-8D show the relative Ki67 or cleaved caspase 3 positive cell number in omacetaxine treated mice compared with control treatment groups. (8E-8G) Omacetaxine suppressed HCC oncoprotein: c-Myc, Beta-catenin and MET protein expression in 50 HCC PDX treated with Omacetaxine vs control.

### DETAILED DESCRIPTION OF THE INVENTION

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

As stated above, HCC is increasing in incidence³⁰. The primary driver of HCC in the USA is the increasing incidence of obesity and fatty liver disease³¹. The only curative strategy is surgical (either resection or liver transplantation)³². Unfortunately, only a minority of patients are surgical candidates, and the rest have very limited chemotherapy options. Sorafenib is a multi-tyrosine kinase inhibitor that was the first drug approved by the FDA for treating HCC that is passed surgical resectability or transplant criteria⁹. Sorafenib, however, has limited benefit for HCC patients (median increase in life span of 9.2 months). Other tyrosine kinase inhibitors were also recently approved (regorafenib, levantinib and cabozantinib). Nivolumab and pembrolizumab (immunotherapy) were also recently approved for HCC following demonstration of efficacy in a subgroup of patients. Last, in May 2019, ramucirumab (monoclonal VEGFR2 antibody) received approval for HCC. While the accumulation of HCC drugs has been very rapid in the last few years, there is still a high demand for effective drugs. It is likely that further research will more accurately provide with patient stratification so that the choice of drugs can be informed in a personalized fashion. Further research will identify what is the patient strata for which omacetaxine could be effective.

The successful establishment of cancer patient derived organoids (PDO) from human HCC has been reported by the inventors^{12, 13}. In addition, the inventors have reported that medium-throughput drug screening in HCC PDO is feasible. Here, the inventors bring further proof that drug screening in HCC PDOs can be utilized for drug discovery or drug repurposing. Patient derived xenografts (PDX) models of human cancer are established as excellent models to recapitulate cancer biology, as well as for drug testing^{28, 29}.

In accordance with some embodiments, the present inventors first identified omacetaxine as a potentially effective HCC therapeutics in HCC PDOs, and the inventors aimed at verifying this finding in a validated confirmatory model. To this end, the inventors have established 2 PDX models from human HCC. Next, the inventors have now shown that indeed, omacetaxine is effective in each of these 2 PDX models. These findings confirm that omacetaxine is likely to be effective in the patients from whom the PDX models were established, and by extension, possibly effective in larger cohorts of HCC patients.

In addition to the successful identification and validation of omacetaxine as a novel therapeutics, the present invention brings further evidence that PDO drug screening is an effective tool that can be incorporated in drug screening strategies. In contrast, drug screening on HCC cell lines has been fraught with multiple false positive findings—i.e., drugs that appeared to be effective in cell lines, only to fail in human clinical trials. The inability to correctly predict effective drugs in pre-clinical trials is partially responsible for the high cost of bringing a new therapeutic to the market. The current invention illustrates HCC PDOs should be integrated in current drug development pipelines, prior to human clinical trials.

As such, in accordance with the following disclosed embodiments, the present invention provides a suppressor of protein translation which is (omacetaxine mepusuccinate, (C₂₉H₃₉NO₉)):

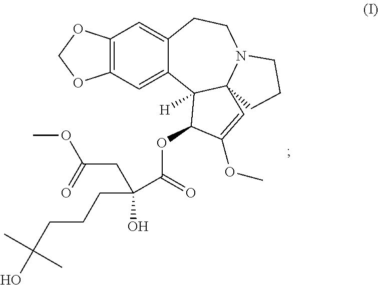 [SMILES: COC(=O)C[C@](O)(CCCC(C)(C)O)C(=O)O[C@@H]1C(OC)=C[C@]23CCCN2CCc2cc4c(cc2[C@H]13)OCO4]

or a pharmaceutically acceptable salt, solvate, analog, derivative or stereoisomer thereof.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “activity” refers to the ability of a gene to perform its function such as Indoleamine 2,3-dioxygenase (an oxidoreductase) catalyzing the degradation of the essential amino acid tryptophan (trp) to N-formyl-kynurenine.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “disease” is meant any condition or disorder that damages or interferes with the normal function of a cell, tissue, or organ. Examples of diseases include cancer.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “express” is meant the ability of a gene to express the gene product including for example its corresponding mRNA or protein sequence (s).

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “obtaining” as in “obtaining an agent” is meant synthesizing, purchasing, or otherwise acquiring the agent.

By “omacetaxine mepusuccinate” means (International nonproprietary name, trade names Synribo), and formerly named as homoharringtonine or HHT. Omacetaxine was originally found in Chinese herbal extracts from the bark of the Chinese plum yew, Cephalotaxus³³. This bark extract was known in traditional Chinese medicine to have anti-cancer activity. This extract was brought to the Western world and characterized in the early 1970's³⁴⁻³⁶. Initial studies in China, performed with unclear combination of active compounds demonstrated activity against AML and CML^{35,36}. More recently, the FDA approved omacetaxine for the treatment of certain subtypes of CML^{37, 38}. The accelerated approval was based on 2 open label trials in CML patients with the T3151 mutations or CML patients who developed resistance or intolerance to at least 2 prior TKIs^{37,38}. Nonetheless, to our knowledge, there have been no Phase II (efficacy) trials of omacetaxine in any solid liver tumors. Furthermore, there have been no reports to date (clinical trials or pre-clinical mouse model or cell line research) to report that omacetaxine is effective in HCC. Therefore, the current study opens the door for further studies that should demonstrate the efficacy of omacetaxine in human clinical trials.

Omacetaxine is a first in class of protein translation inhibitors²⁰. The drug does not appear to target a specific protein, but rather inhibits overall protein translation. Overall inhibition of protein translation affects predominantly proteins with rapid turnover (short half-life)²⁰. Omacetaxine is efficacious in CML due to blockade of oncoproteins that have short half-lives^{26,42}.

In accordance with some embodiments of the present invention, the inventors have first shown that omacetaxine affects the global protein translation in PDOs. To our knowledge, this is the first study to report these findings. Furthermore, the inventors have shown that several oncoproteins (C-MYC, MET, beta-catenin, XIAP, and Cyclin D1) display diminished levels following treatment with omacetaxine both in PDOs as well as in PDX models in vivo. Last, a key aspect of any drug in this class appears to be the transient use of such a drug as to not affect proteins that have longer half-lives²⁰. Further efforts to position omacetaxine for HCC treatment should take into consideration the need for its transient utilization.

Omacetaxine is a pharmaceutical drug substance that is indicated for treatment of chronic myeloid leukemia (CML). It is derived from a natural product first discovered in Cephalotaxus harringtonii, now manufactured by hemi-synthesis. The U.S. FDA approved it in October 2012 for the treatment of adult patients with CML with resistance and/or intolerance to two or more tyrosine kinase inhibitors (TKIs). It has the chemical structure of Formula I above, or a pharmaceutically acceptable salt, solvate, analog, derivative or stereoisomer thereof. It inhibits protein translation by preventing the initial elongation step of protein synthesis.

Omacetaxine interacts with the ribosomal A-site and prevents the correct positioning of amino acid side chains of incoming aminoacyl-tRNAs. Omacetaxine acts only on the initial step of protein translation and does not inhibit protein synthesis from mRNAs that have already commenced translation.

Analogs and derivatives of omacetaxine are known in the art. Examples of such analogs and derivatives can be found in International Patent Publication No. WO2016182850A1, and incorporated by reference herein.

By “polypeptide,” “peptide” and “protein” is meant terms that may be used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

By “prevent,” “preventing,” “prevention,” “prophylactic treatment” and the like is meant reducing the probability of developing a disorder or condition in a subject, who does not have, but is at risk of or susceptible to developing a disorder or condition.

By “ranges” is meant all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “reduces” is meant a negative alteration such as lowering the amount or concentration of something by at least 10%, 25%, 50%, 75%, or 100%, as an example.

By “reference” is meant a standard or control conditions such as a sample (human cells) or a subject that is a free, or substantially free, of an agent such as one or more inhibitors of IDO1 and/or a vaccine.

By “sensitivity” is meant the percentage of subjects with a particular disease.

By “solid cancer” or “solid tumor” is meant an abnormal mass of tissue that are malignant. Different types of solid tumors are named for the type of cells that form them. Examples of solid cancers are adenocarcinomas, sarcomas, carcinomas, and lymphomas.

By “specificity” is meant the percentage of subjects correctly identified as not having a particular disease i.e., normal or healthy subjects.

By “subject” is meant any individual or patient to which the method described herein is performed. Generally, the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

By “treat,” treating,” “treatment,” is meant reducing or ameliorating a disorder and/or symptoms associated therewith. It will be appreciated that, although not precluded, treating a disorder or condition does not require that the disorder, condition or symptoms associated therewith be completely eliminated. Treating is synonymous with the use of the compositions and methods in the treatment of a liver cancer or tumor in a subject.

In an embodiment, the term “administering” means that the compounds of the present invention are introduced into a subject, preferably a subject receiving treatment for a proliferative disease, and the compounds are allowed to come in contact with the one or more disease related cells or population of cells in vivo.

As defined herein, in another embodiment, the term “contacting” means that the one or more compounds of the present invention are introduced into a sample having at least one cancer cell and appropriate enzymes or reagents, in a test tube, flask, tissue culture, chip, array, plate, microplate, capillary, or the like, and incubated at a temperature and time sufficient to permit binding and uptake of the at least one compound to the cancer cell. Methods for contacting the samples with the compounds, and other specific binding components are known to those skilled in the art, and may be selected depending on the type of assay protocol to be run. Incubation methods are also standard and are known to those skilled in the art.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Such treatment (surgery and/or chemotherapy) will be suitably administered to subjects, particularly humans, suffering from, having, susceptible to, or at risk for cancer or disease, disorder, or symptom thereof. Determination of those subjects “at risk” can be made by any objective or subjective determination by a diagnostic test or opinion of a subject or health care provider (e.g., genetic test, enzyme or protein marker, a marker (as defined herein), family history, and the like).

The term “chemotherapeutic agent” as well as words stemming therefrom, as used herein, generally includes pharmaceutically or therapeutically active compounds that work by interfering with DNA synthesis or function in cancer cells. Based on their chemical action at a cellular level, chemotherapeutic agents can be classified as cell-cycle specific agents (effective during certain phases of cell cycle) and cell-cycle nonspecific agents (effective during all phases of cell cycle). Without being limited to any particular example, examples of chemotherapeutic agents can include alkylating agents, angiogenesis inhibitors, aromatase inhibitors, antimetabolites, anthracyclines, antitumor antibiotics, monoclonal antibodies, platinums, topoisomerase inhibitors, and plant alkaloids.

In a further embodiment, the compositions and methods of the present invention can be used in combination with one or more additional therapeutically active agents which are known to be capable of treating conditions or diseases discussed above. For example, the compositions of the present invention could be used in combination with one or more known therapeutically active agents, to treat a proliferative disease. Non-limiting examples of other therapeutically active agents that can be readily combined in a pharmaceutical composition with the compositions and methods of the present invention are enzymatic nucleic acid molecules, allosteric nucleic acid molecules, antisense, decoy, or aptamer nucleic acid molecules, antibodies such as monoclonal antibodies, small molecules.

An active agent and a biologically active agent are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se as well as pharmaceutically acceptable, pharmacologically active salts, esters, amides, prodrugs, metabolites, analogs etc. The active agent can be a biological entity, such as a virus or cell, whether naturally occurring or manipulated, such as transformed.

The biologically active agent may vary widely with the intended purpose for the composition. The term active is art-recognized and refers to any moiety that is a biologically, physiologically, or pharmacologically active substance that acts locally or systemically in a subject. Examples of biologically active agents, that may be referred to as “drugs”, are described in well-known literature references such as the Merck Index, the Physicians' Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment.

The inventors discovered that omacetaxine is effective in a large cohort of HCC PDOs, as well as in vivo in patient derived xenograft (PDX) models and they have characterized its mechanism of action in vitro as well as in vivo.

In accordance with a first embodiment, the present invention provides a method for treating a solid cancer or tumor in a subject comprising the steps of administering to the subject an effective amount of a suppressor of protein translation to the subject. In some embodiments, the suppressor or inhibitor of protein translation binds the ribosomal A-site and prevents the correct positioning of amino acid side chains of incoming aminoacyl-tRNAs.

In accordance with a second embodiment, the present invention provides a method for treating a liver tumor in a subject comprising the steps of administering to the subject an effective amount of a suppressor of protein translation to the subject.

In accordance with a third embodiment, the present invention provides a method for treating a liver tumor in a subject comprising the steps of administering to the subject an effective amount of omacetaxine to the subject.

In accordance with a fourth embodiment, the present invention provides a method for treating a liver tumor in a subject comprising the steps of administering to the subject an effective amount of a suppressor of protein translation and at least one additional biologically active agent to the subject.

In accordance with a fifth embodiment, the present invention provides a method for treating a liver tumor in a subject comprising the steps of administering to the subject an effective amount of a suppressor of protein translation and at least one additional chemotherapeutic agent to the subject.

In certain embodiments, the level to which a suppressor of protein translation decreases protein expression may be any level so long as it provides amelioration of at least one symptom of the liver cancer, including HCC. The level of protein translation may decrease by at least 2, 3, 4, 5, 10, 25, 50, 100, 1000, or more fold expression compared to the level of expression in a standard, in at least some cases. An individual may monitor expression levels of proteins using standard methods in the art, such as northern assays, for example.

An individual known to have HCC, suspected of having HCC, or at risk for having HCC may be provided an effective amount of a suppressor of protein translation, including Omacetaxine mepusuccinate salt, solvate, derivative, or stereoisomer thereof. Those at risk for HCC may be those individuals having one or more genetic factors, may be of advancing age, and/or may have a family history, for example.

In particular embodiments of the disclosure, an individual is given an agent for HCC therapy in addition to the one or more suppressor of protein translation including Omacetaxine mepusuccinate salt, solvate, derivative, or stereoisomer thereof. Such additional therapy may include one or more chemotherapies, for example. When combination therapy is employed with one or more suppressor of translation, the additional therapy may be given prior to, at the same time as, and/or subsequent to the one or more suppressor of translation.

Pharmaceutical Preparations

Pharmaceutical compositions used in the methods of the present invention comprise an effective amount of one or suppressors of protein translation such as omacetaxine mepusuccinate salt, solvate, analog, derivative, or stereoisomer thereof, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate.

The preparation of a pharmaceutical composition that comprises at least one suppressors of protein translation such as omacetaxine mepusuccinate salt, solvate, derivative, or stereoisomer thereof, or additional active ingredient will be known to those of skill in the art in light of the present disclosure, as exemplified by Remington: The Science and Practice of Pharmacy, 22^{st} Ed. Pharmaceutical Press, 2012, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's above). Except insofar as any conventional carrier is incompatible with the active ingredient, its use in the pharmaceutical compositions is contemplated.

The suppressor of protein translation such as omacetaxine mepusuccinate salt, solvate, derivative, or stereoisomer thereof, may comprise different types of carriers depending on whether it is to be administered in solid, liquid or aerosol form, and whether it need to be sterile for such routes of administration as injection. The present compositions can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art.

Further, in accordance with the present invention, the compositions used in the methods of the present invention suitable for administration can be provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with some embodiments of the present invention, the compositions used in the inventive methods can be combined with the carrier in any convenient and practical manner, i.e., by solution, suspension, emulsification, admixture, encapsulation, absorption and the like. Such procedures are routine for those skilled in the art. In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the methods of the present invention may involve the use of a pharmaceutical lipid vehicle composition that include a suppressor of protein translation such as omacetaxine mepusuccinate salt, solvate, analog, derivative, or stereoisomer thereof, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the suppressor of protein translation such as omacetaxine mepusuccinate salt, solvate, analog, derivative, or stereoisomer thereof, may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition used in the methods of the present invention administered to a subject can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will determine the concentration of active ingredient(s) in a composition and appropriate dose(s) for the individual subject. In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

Alimentary Compositions and Formulations

In one embodiment of the present disclosure, a suppressor of protein translation such as omacetaxine mepusuccinate salt, solvate, analog, derivative, or stereoisomer thereof (an example of an active compound), is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, they may be enclosed in hard- or soft-shell gelatin capsule, they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

Additional formulations that are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Parenteral Compositions and Formulations

In further embodiments, suppressors of protein translation, such as omacetaxine mepusuccinate salts, solvates, analogs, derivatives, or stereoisomers thereof, may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety).

In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion. Some variation in dosage will necessarily occur depending on the condition of the subject being treated. The person responsible for administration will, in any event, determine the appropriate dose for the individual subject.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

Kits of the Disclosure

Any of the compositions described herein may be comprised in a kit. In a non-limiting example, a suppressor of protein translation (for example, Omacetaxine mepusuccinate salt, solvate, derivative, or stereoisomer thereof) may be comprised in a kit. The kits may comprise a suitably aliquoted of a suppressor of protein translation and, in some cases, one or more additional agents. The component(s) of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the suppressor of protein translation and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. The suppressor of protein translation composition(s) may be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit. However, the components of the kit may be provided as dried powder(s). When reagents and/or components are provided as a dry powder, the powder can be reconstituted by the addition of a suitable solvent. It is envisioned that the solvent may also be provided in another container means.

### EXAMPLES

HCC PDOs were established and validated.

The inventors established 10 HCC PDO lines¹³. In a similar fashion, the inventors established an additional 30 HCC PDO lines, for a total of 40 lines. For the establishment of these lines, the inventors have utilized resection tissue from surgery (20 patients), as well as from biopsy material (1 patient). For some patients, the tumor mass was sufficiently large to allow establishment of more than 1 HCC line. As the inventors have previously shown, in large HCC tumors, geographically distinct areas display genetic as well as drug-response heterogeneity¹³. Clinical information and histopathological information of the PDOs are summarized in Table 1.

TABLE 1
Clinical information of PDO lines

| | Sample | PDO | | | | | | Biopsy/ | Liver |
|---|---|---|---|---|---|---|---|---|---|
| | Name | lines | Sex | Race | Age | Stage | Differentiation | Resection | disease |
| | HCC 24 | 1 | m | w | 24 | PT1N0MX | Moderate | Resection | PSC |
| | HCC 25 | 3 | m | w | 73 | PT1N0MX | Moderate | Resection | |
| | HCC 26 | 7 | f | w | 71 | PT3bNxMx | Moderate | Resection | HCV |
| | HCC 30 | 1 | m | b | 63 | PT1NXMx | Poor | Resection | |
| | HCC 34 | 1 | m | b | 62 | PT2NxMx | Moderate | Resection | HCV |
| | HCC 35 | 1 | f | w | 80 | PT2NxMx | Poor | Resection | |
| | HCC 36 | 1 | m | w | 83 | PT2NxMx | Moderate | Resection | |
| | HCC 37 | 1 | m | o | 65 | PT1N0MX | Moderate | Resection | HBV |
| | HCC 38 | 1 | f | w | 89 | PT2NxMX | Poor | Resection | |
| | HCC 40 | 1 | f | b | 65 | PT2NxMX | Moderate | Resection | HCV |
| | HCC 50 | 1 | f | w | 65 | PT2N0M0 | Moderate | Resection | |
| | HCC 57 | 1 | f | w | 77 | PT2NXM1 | Moderate | Biopsy | |
| | HCC 59 | 1 | m | b | 62 | PT2NxMX | Moderate | Resection | |
| | HCC 65 | 2 | f | b | 60 | PT2N0MX | Moderate | Resection | HBV |
| | HCC 67 | 4 | f | b | 42 | PT2NxMx | Moderate | Resection | HBV |
| | HCC 71 | 4 | f | w | 76 | PT3N0MX | Moderate | Resection | |
| | HCC 76 | 1 | m | w | 28 | PT3N0M1 | Well | Resection | |
| | HCC 77 | 1 | m | w | 69 | PT1bN1M1 | Poor | Resection | |
| | HCC 80 | 4 | f | b | 64 | PT2NxMx | Moderate | Resection | HCV/HBV |
| | HCC 83 | 3 | f | w | 72 | PT2NxMx | Moderate | Resection | |
| Total | 20 | 40 | | | | | | | |

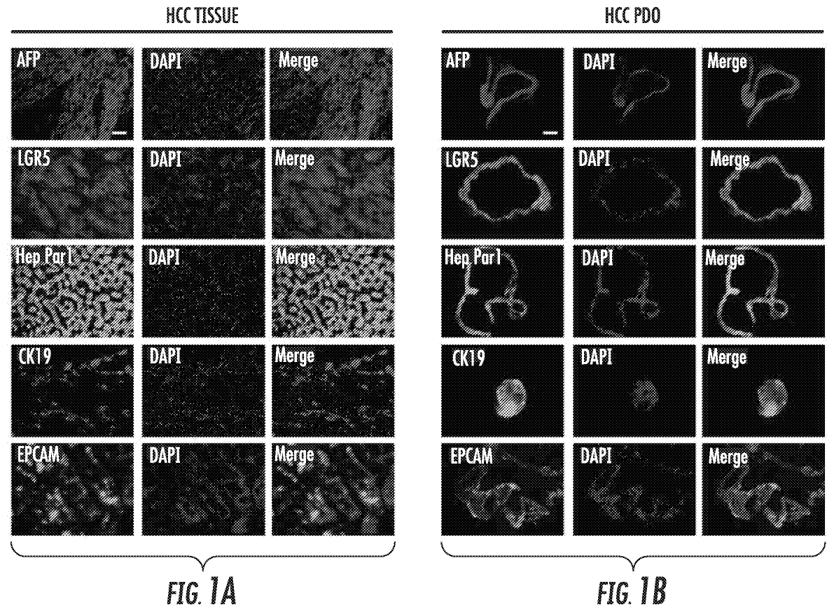

Staining for HCC markers (hepatocyte paraffin 1—HepPar1; alfa feto protein—AFP), as well as stem cell markers (leucine rich repeat containing G protein coupled receptor 5—LGR5), cholangiocyte and hepatocyte marker (cytokeratin 19-CK19), epithelial marker (epithelial cellular adhesion molecule—EPCAM) was performed. Representative HCC tissues, as well as matched PDO, are shown in FIG. 1A.

### Example 1

Drug Screening Identifies Omacetaxine as a Putative Anti-HCC Agent.

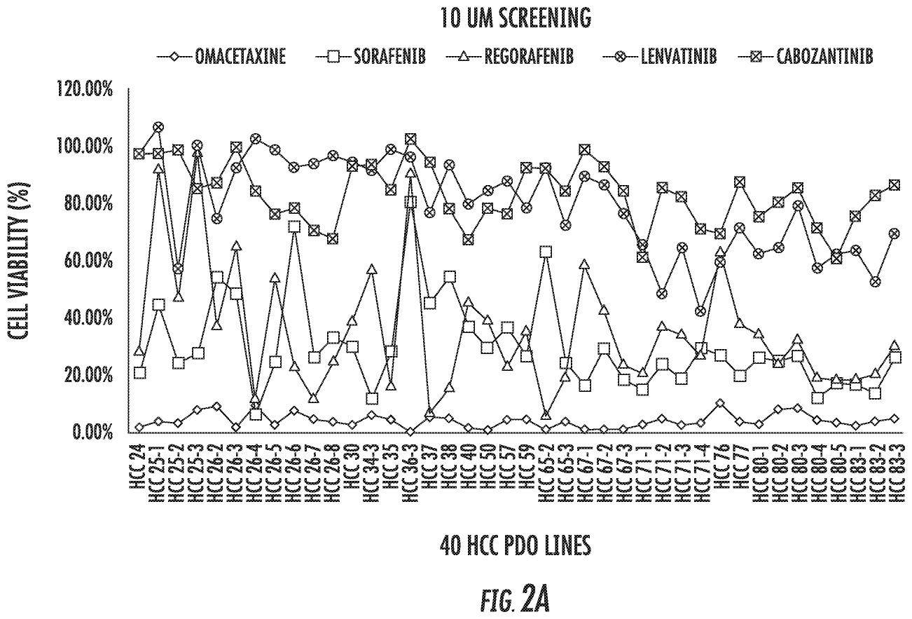

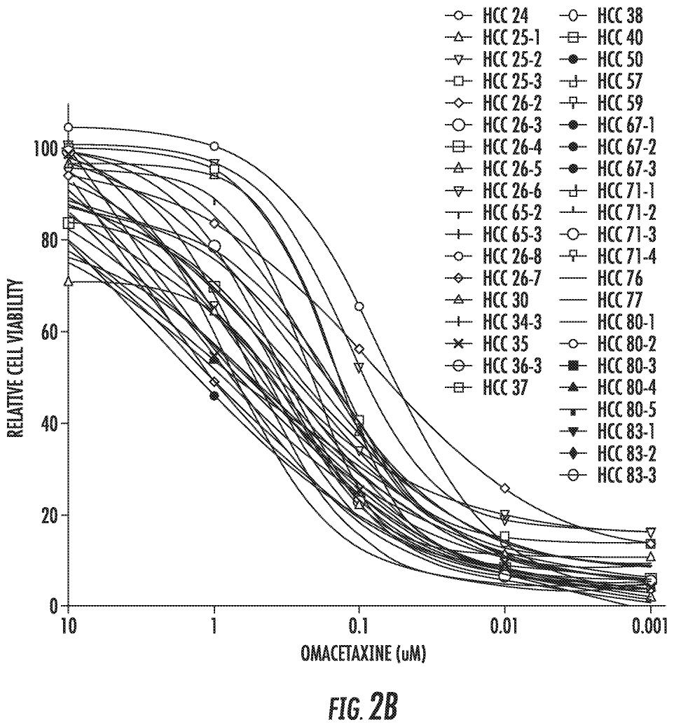

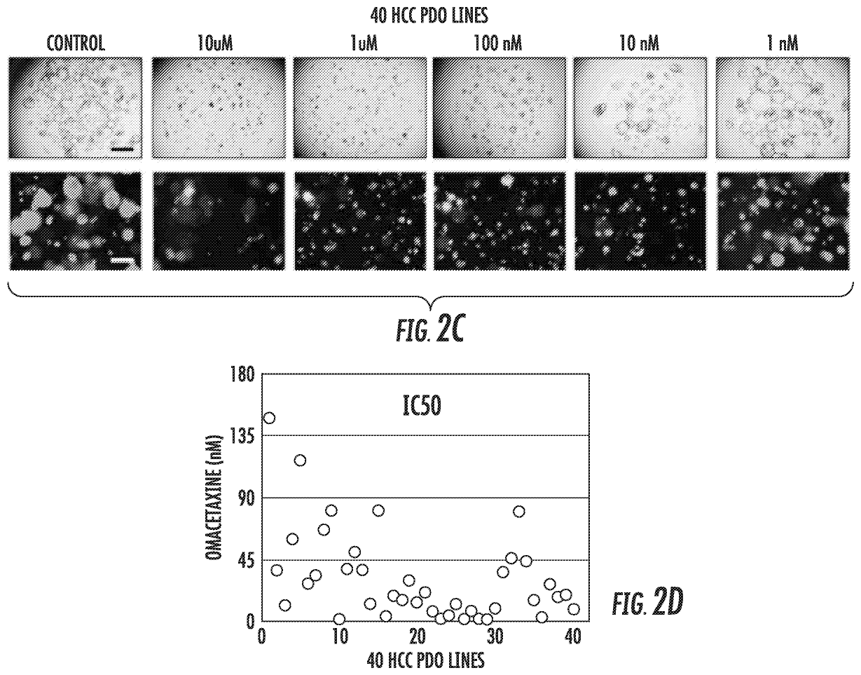

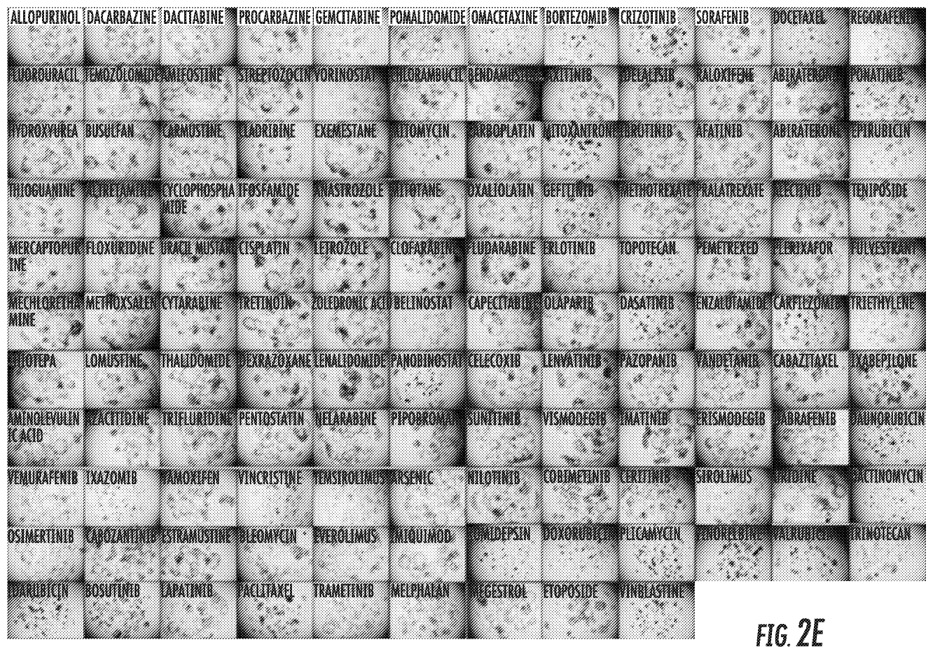

After successful establishment of the 40 HCC PDO lines from 20 HCC patients, the inventors performed drug screening with a panel of 129 anti-cancer drugs, similar to our published data¹³. As readout of drug effectiveness, cell viability at 96 hours (h) was measured. White light microscopy images of one of the HCC PDO lines across 129 drugs was prepared. In FIG. 2E, each panel represents the image collected at 96 hours after treatment with each drug, at a concentration of 10 μM. As seen in the figure, certain drugs, such as bortezomib, induce cell destruction while others, such as sorafenib, allow unrestricted organoid growth. To quantify survival, we implemented CellTiter-Glo, a luminescent cell viability assay. A total of 129 readings (one for each drug) was obtained for each of the 40 PDO lines, for a total of 4,644 data points. For each individual HCC PDO, drugs that inhibited the growth by ≥50% at 96 h at the screening concentration of 10 μM were considered effective and chosen for further consideration. The inventors found that omacetaxine, a protein translation inhibitor, was effective across all 40 HCC PDO lines, with an average HCC PDO survival of only 4.2%. Next, the inventors contrasted the survival of the 4 HCC PDO lines when treated with omacetaxine versus current FDA approved drugs for HCC. The inventors found that, in their in vitro PDO system, omacetaxine was significantly more effective than first line HCC drugs (sorafenib and lenvatinib) and second line HCC drugs (regorafenib and cabozantinib, FIG. 2A).

### Example 2

Omacetaxine is Effective at Nanomolar Concentrations in HCC In Vitro.

The inventors tested omacetaxine at decreasing concentrations (10 μM, 1 μM, 100 nM, 10 nM and 1 nM) to calculate the half-maximal inhibitory concentration (IC₅₀). As shown in FIG. 2C, and further validating the present assay, omacetaxine displays dose response (decreasing concentrations result in less dead cells—red in the figure—and more live cells—green in the figure). FIG. 2B displays the IC₅₀ curves for each of the 40 HCC PDOs. While there was variability noted—which is to be expected as an expression of functional heterogeneity—the curves demonstrate that all 40 HCC PDOs are sensitive to omacetaxine at nanomolar concentrations. The calculated average IC₅₀ across all 40 HCC PDO lines was 35.2 nM. The IC₅₀ value for each of the 40 HCC PDO lines can be found in Table 2. FIG. 2D displays the values for each of the HCC PDO lines.

TABLE 2
IC50 of 40 PDO lines from 20 HCC patients.

| PDO lines | HCC 24 | HCC 25-1 | HCC 25-2 | HCC 25-3 | HCC 26-2 | HCC 26-3 | HCC 26-4 | HCC 26-5 | HCC 26-6 | HCC 26-7 |
|---|---|---|---|---|---|---|---|---|---|---|
| IC50 (nM) | 148.5 | 37.35 | 11.73 | 60.15 | 117.8 | 27.84 | 33.53 | 66.74 | 80.75 | 1.407 |
| PDO lines | HCC 26-8 | HCC 30 | HCC 34-3 | HCC 35 | HCC 36-3 | HCC 37 | HCC 38 | HCC 40 | HCC 50 | HCC 57 |
| IC50 (nM) | 37.83 | 50.59 | 37.7 | 13.07 | 80.72 | 3.519 | 18.53 | 15.75 | 29.84 | 14.12 |
| PDO lines | HCC 59 | HCC 65-2 | HCC 65-3 | HCC 67-1 | HCC 67-2 | HCC 67-3 | HCC 71-1 | HCC 71-2 | HCC71-3 | HCC 71-4 |
| IC50 (nM) | 21.21 | 7.402 | 2.26 | 4.027 | 12.84 | 1.674 | 7.29 | 2.053 | 1.511 | 9.985 |
| PDO lines | HCC 76 | HCC 77 | HCC 80-1 | HCC 80-2 | HCC 80-3 | HCC 80-4 | HCC 80-5 | HCC 83-1 | HCC 83-2 | HCC 83-3 |
| IC50 (nM) | 35.54 | 46.11 | 80.11 | 44.05 | 15.66 | 3.176 | 27.65 | 17.71 | 19.19 | 8.846 |

Based on these data, the inventors concluded that omacetaxine demonstrates potential as a novel HCC therapeutics and decided to elucidate its effects and mechanisms of action.

### Example 3

Omacetaxine Represses Growth and Increases Apoptosis in HCC PDOs.

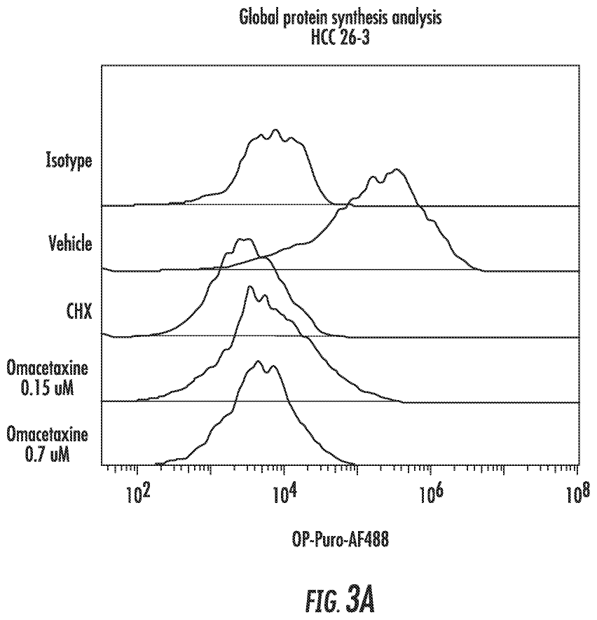

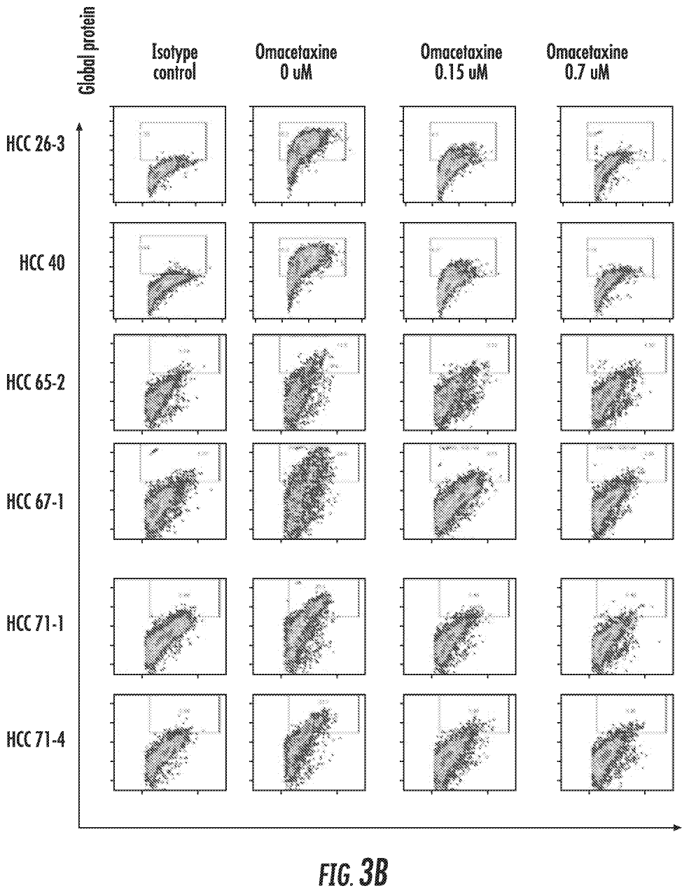

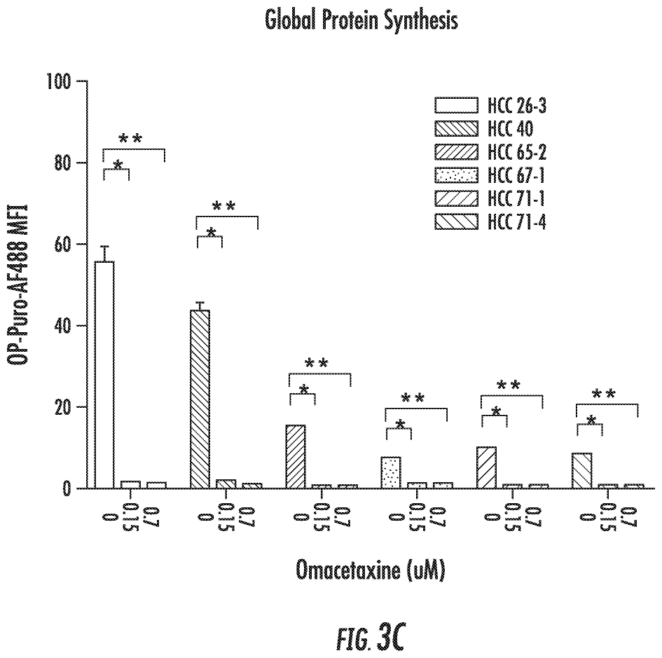

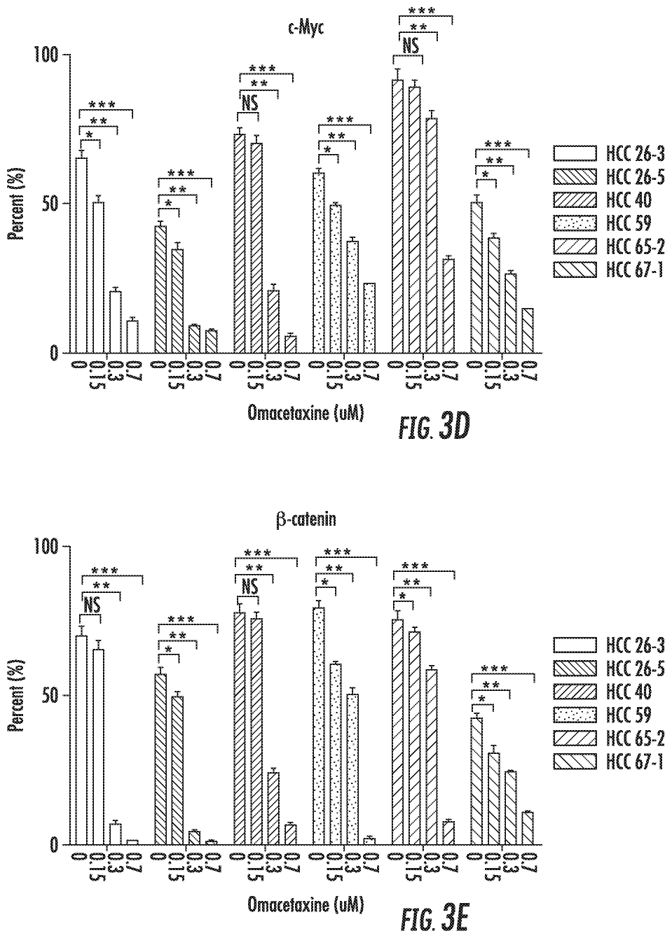

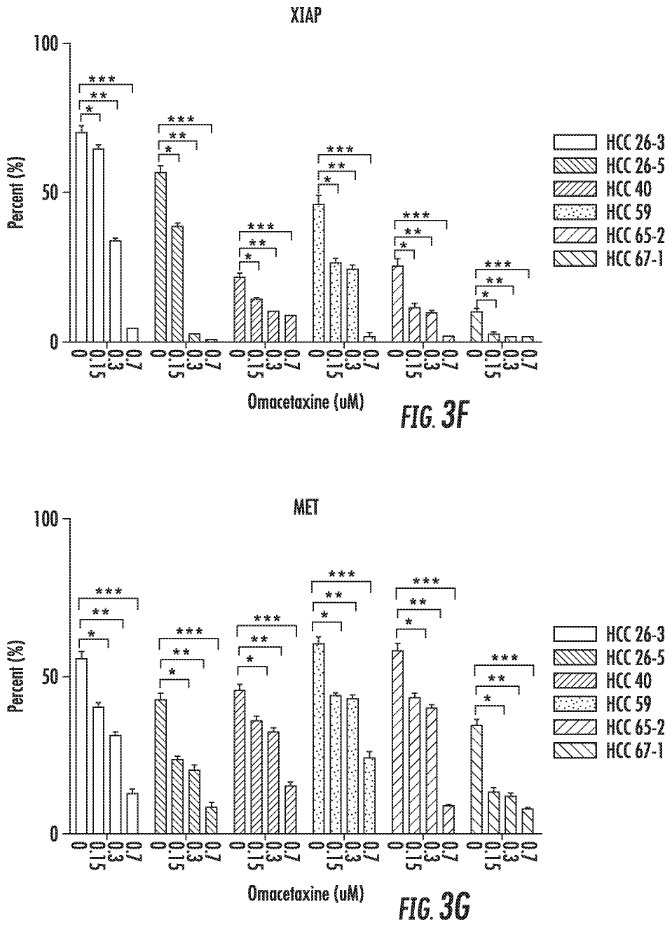

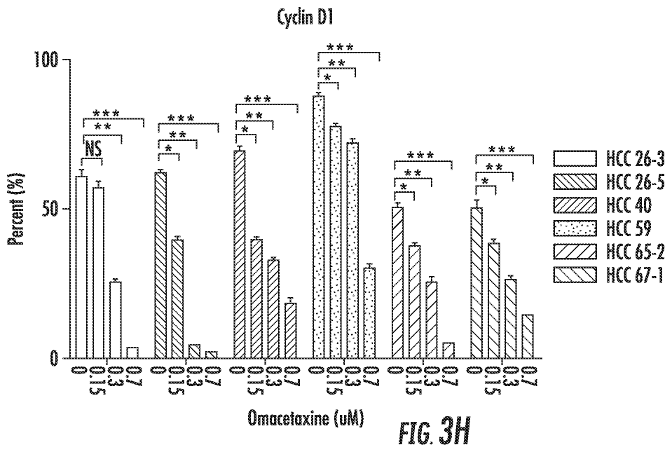

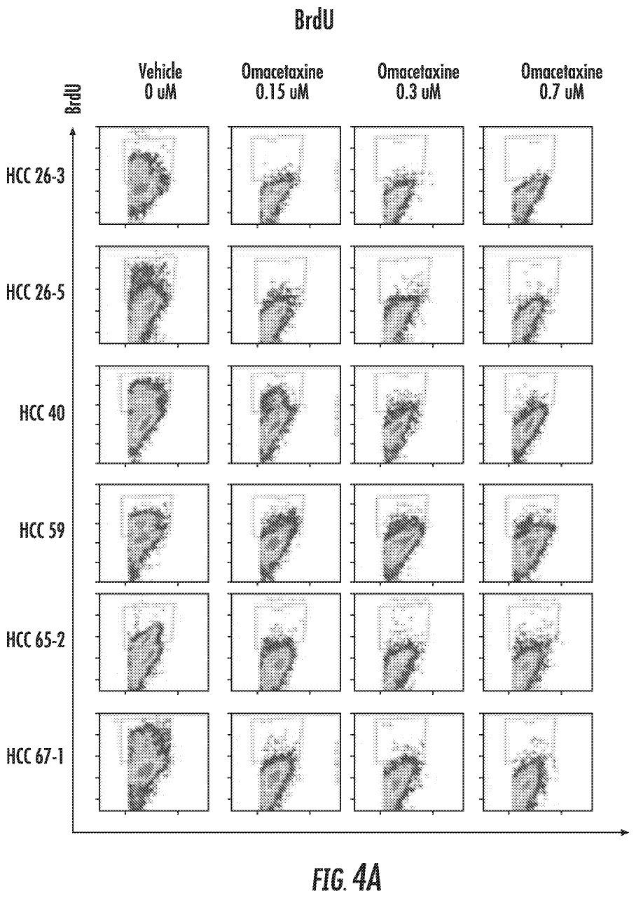

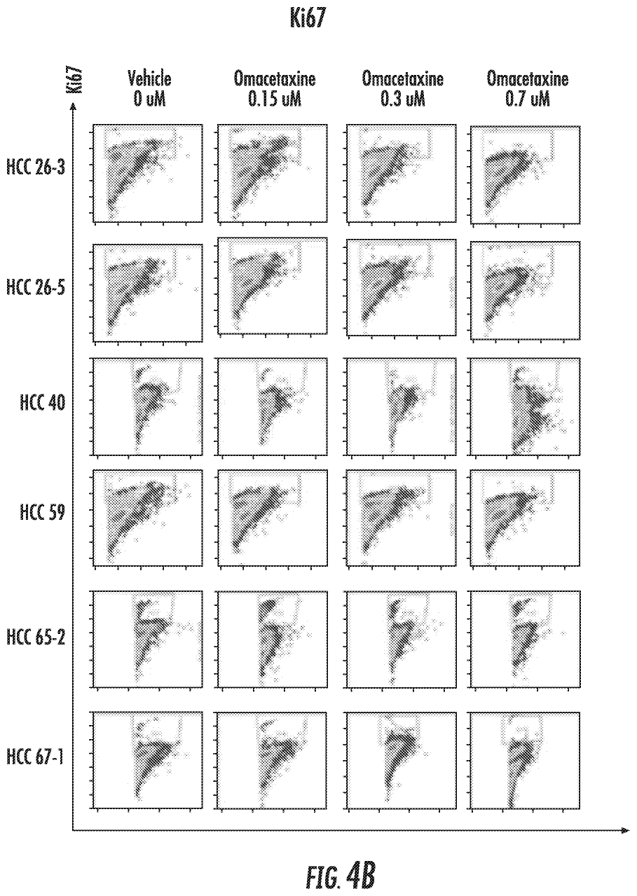

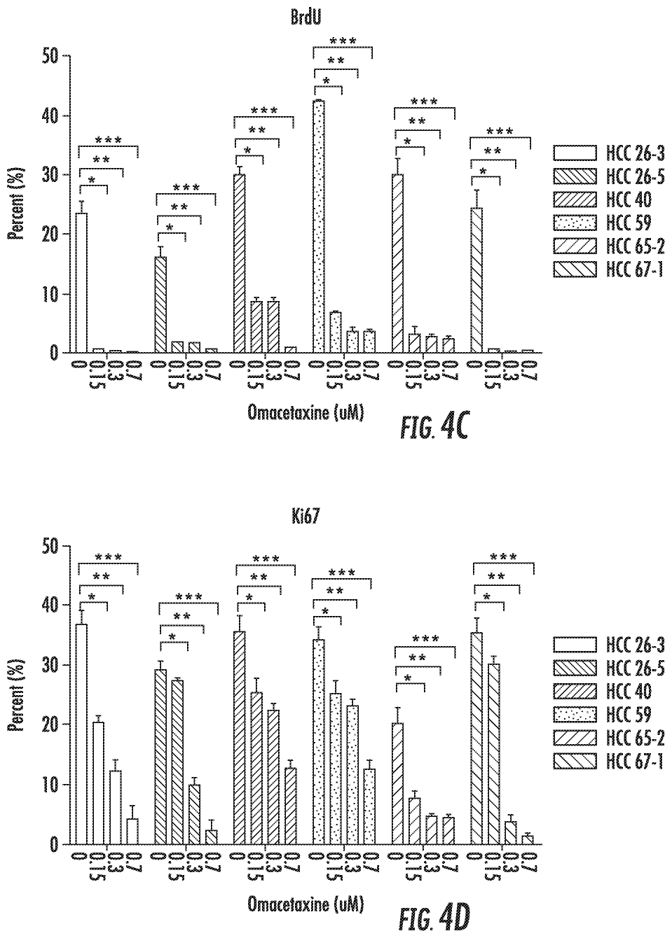

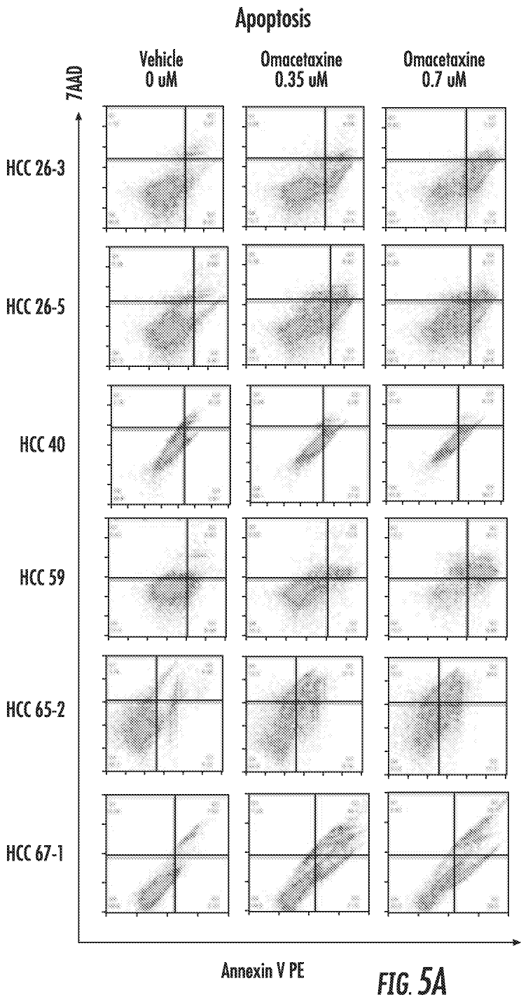

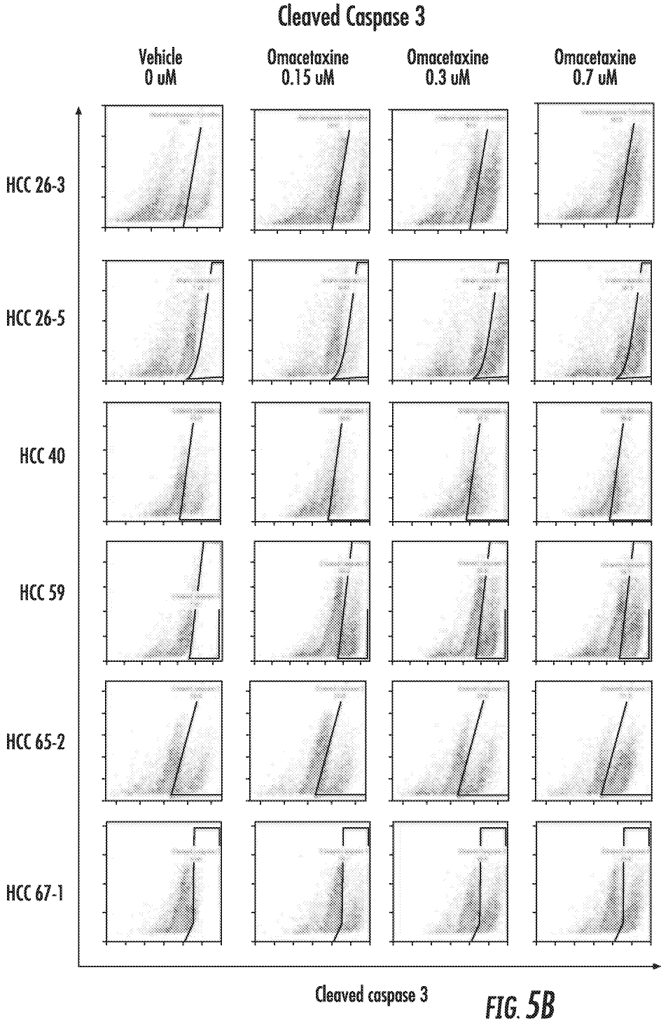

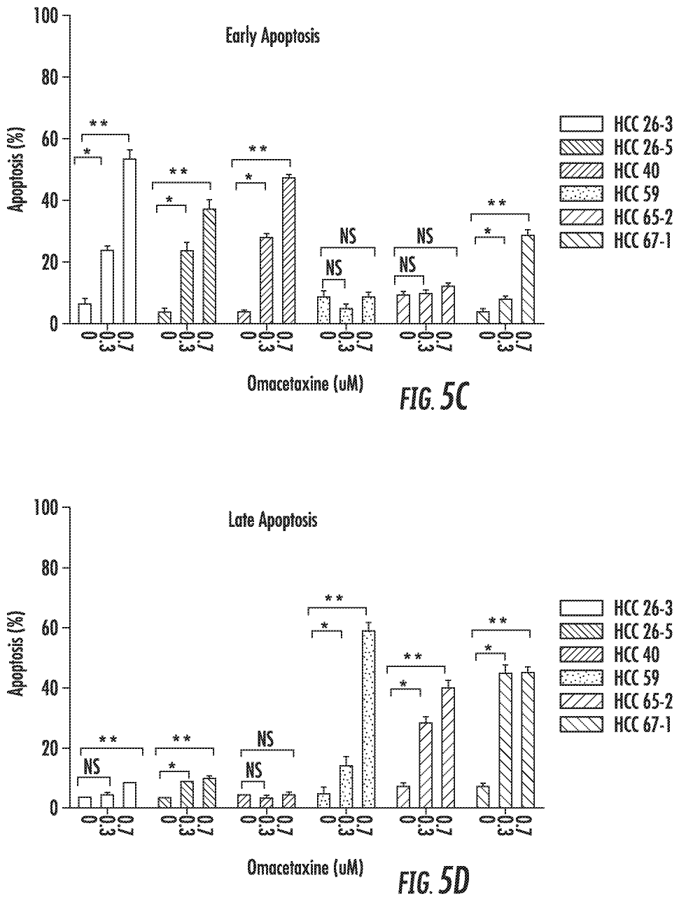

The effects on proliferation were assessed to elucidate the omacetaxine mechanisms of action. FACS analysis of bromodeoxyuridine (BrdU) incorporation demonstrated that omacetaxine inhibited incorporation of this synthetic nucleoside in newly synthetized DNA during the S phase. By extension, the implication is that omacetaxine inhibits cancer cell proliferation in each of the 6 HCC PDO lines tested (FIGS. 3A and 3C). To further understand the effects of omacetaxine on cancer cell proliferation and cell cycle, the inventors performed FACS analysis of Ki67 protein. Ki-67 is a nuclear protein expressed in proliferating cells (in all phases of the cell cycle except G0)^{22,23}. Furthermore, the higher expression of Ki67 has been linked to poorer disease free survival and overall survival in HCC²⁴. As shown in FIGS. 3B and 3D, omacetaxine reduces the expression of Ki67 in all 6 HCC PDO lines tested. Next, the inventors performed a cell cycle analysis to determine at which point the cells arrest. As shown in FIG. 4, omacetaxine induces G0/G1 arrest that likely explains the effects of omacetaxine on cell proliferation noted above. The effects of omacetaxine on G0/G1 arrest, as shown in the figure, appear to be dose-dependent. One of the inventors' previous studies demonstrated that liver cancers (including HCCs) display functional heterogeneity (as measured through drug responses)¹³. However, omacetaxine displays near uniform effectiveness in all 38 HCC PDO lines analyzed here, suggesting that multiple mechanisms of action are possible. Therefore, the inventors tested alternative and complementary mechanisms of cell death, such as apoptosis. Flow cytometry based analysis of Annexin V and 7-Aminoactinomycin D (7AAD) staining in 6 HCC PDO lines demonstrated that either early apoptosis, or late apoptosis, or both were increased in each of these lines (FIGS. 5A, 5C and 5D). As caspase 3 is a key molecule in apoptosis, the inventors measured its activated (cleaved) fragment. Indeed, cleaved caspase 3 was induced in each of the 6 HCC PDO lines tested (FIGS. 5B and 5E).

### Example 4

Omacetaxine Inhibits Global Protein Synthesis.

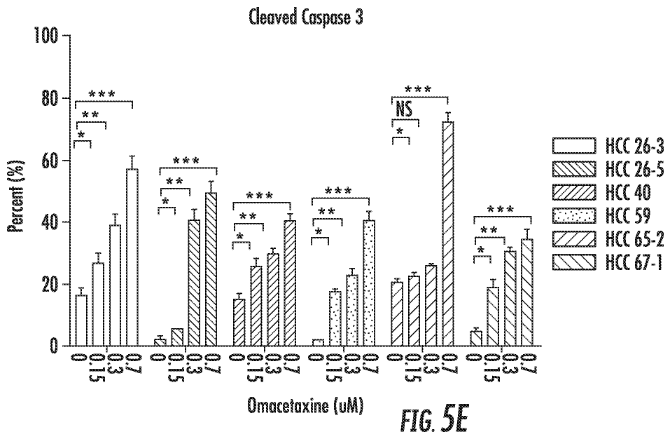

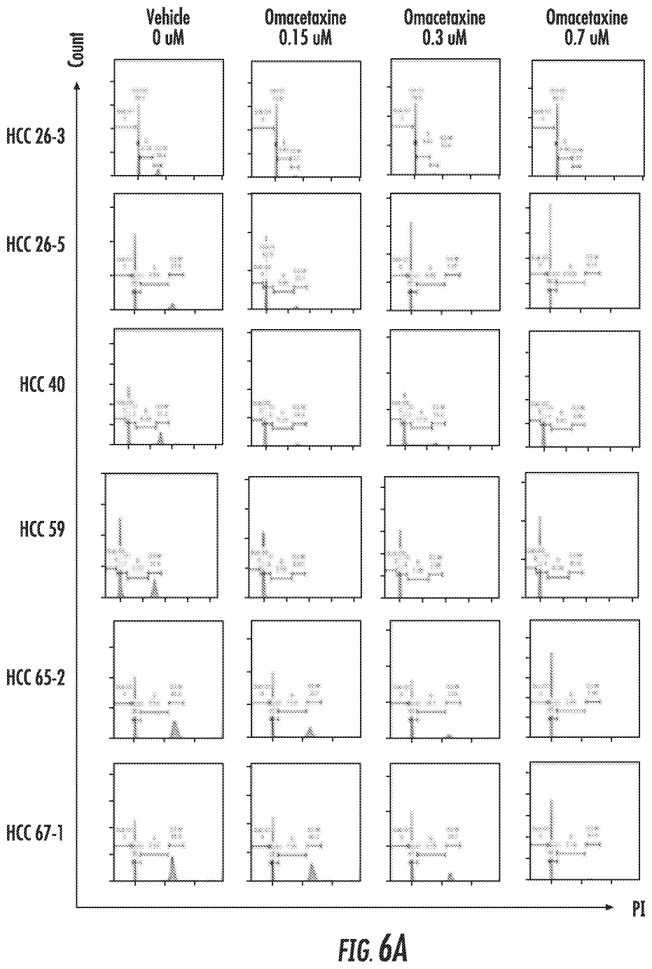

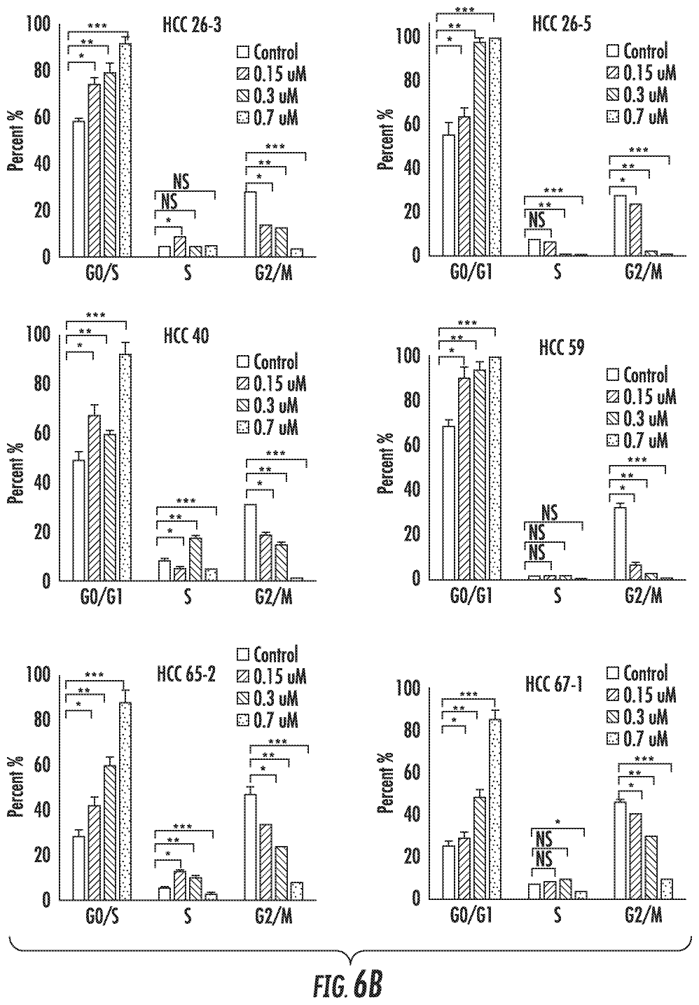

Previous studies showed that omacetaxine inhibits protein synthesis via inhibiting the first peptide bond formation in the synthesis of polypeptides^{20,25}. In acute myelogenous leukemia (AML), short-lived proteins (high synthesis rate) are affected preferentially by global protein synthesis inhibition by omacetaxine¹⁷. Furthermore, certain AML subtypes (such as FLT3-ITD AML) appear to become addicted to a high protein synthesis rate, which explains (at least in part) their sensitivity to omacetaxine¹⁷. Without being limited to any particular mechanism, the inventors hypothesize that the effectiveness of omacetaxine in HCC PDOs, resulting in reduced proliferation and increased apoptosis as noted above, may be explained at least in part by global protein synthesis inhibition. Furthermore, global protein synthesis inhibition may affect preferentially short-lived proteins and cells that depend on high synthesis of short half-life proteins. To test this hypothesis, relative protein synthesis rates from 6 HCC PDOs were measured by 0-Propargyl-Puromycin (OP-Puro) incorporation assay (EZClick™, Global Protein Synthesis Assay, FIG. 6A). Treatment with CHX was used as positive control, and 0.1% DMSO was used as negative control, as per manufacturer's recommendations. FIG. 6B shows the fluorescence-activated cell sorting (FACS) plots for each of the 6 HCC PDOs when treated with isotype control, negative control, as well as omacetaxine at 150 nM and 700 nM. FIG. 6C demonstrates that omacetaxine drastically inhibited global protein synthesis at each of the 2 concentrations (statistically significant). Of interest, a concentration of 700 nM did not induce any further inhibition of protein synthesis when compared with the 150 nm concentration. Taken together with the observation that the average IC₅₀ of omacetaxine across all 40 HCC PDOs is 35.2 nM, the conclusion is that, at least in part, the effectiveness of omacetaxine in HCC PDOs is explained by inhibition of protein synthesis.

### Example 5

Omacetaxine Inhibits Select Oncoprotein Synthesis in HCC.

Select oncoproteins, such as MYC, beta-catenin, Cyclin D1, XIAP and MET are short half-life proteins^{20, 26, 27}. Based on data showing omacetaxine inhibits global protein synthesis (FIGS. 6A-6C), the inventors advanced the hypotheses that (a) omacetaxine inhibits short half-life proteins in HCC PDOs and (b) this inhibition may be part of the mechanism of action. To test these hypotheses, 6 HCC PDO lines were treated with omacetaxine and the levels of these 5 oncoproteins were assayed with FACS at 96 h (FIGS. 6D-6H). The inventors found that each of these oncoproteins (MYC, beta-catenin, Cyclin D1, XIAP and MET) were inhibited by omacetaxine.

In conclusion, not only does omacetaxine inhibit global protein synthesis in HCC, however, the level of specific short half-life proteins is decreased. These short half-life proteins are known oncoproteins and it is likely that omacetaxine acts, at least in part, through the downregulation of these proteins.

### Example 6

Omacetaxine Represses Cancer Growth and Increases Survival in PDX Models of HCC.

Patient derived xenograft (PDX) models of human cancer are well documented to recapitulate primary tumor biology, as well as response to treatment^{28, 29}. Two PDX models from HCC patients were established in the inventors' lab with the purpose of creating a platform for further validation of drug leads. The first model, PDX26-3, was established by subcutaneous injection of HCC PDO cells into NOD SCID-IL2R-γ chain-deficient mice (NSG, Jackson Lab). More specifically, PDX26-3 is established from PDO26-3 cells at passage 3. After the initial PDX establishment, the model is perpetuated by implantation of pieces of tumors from current generation PDX mice into the next (as described in Materials and Methods).

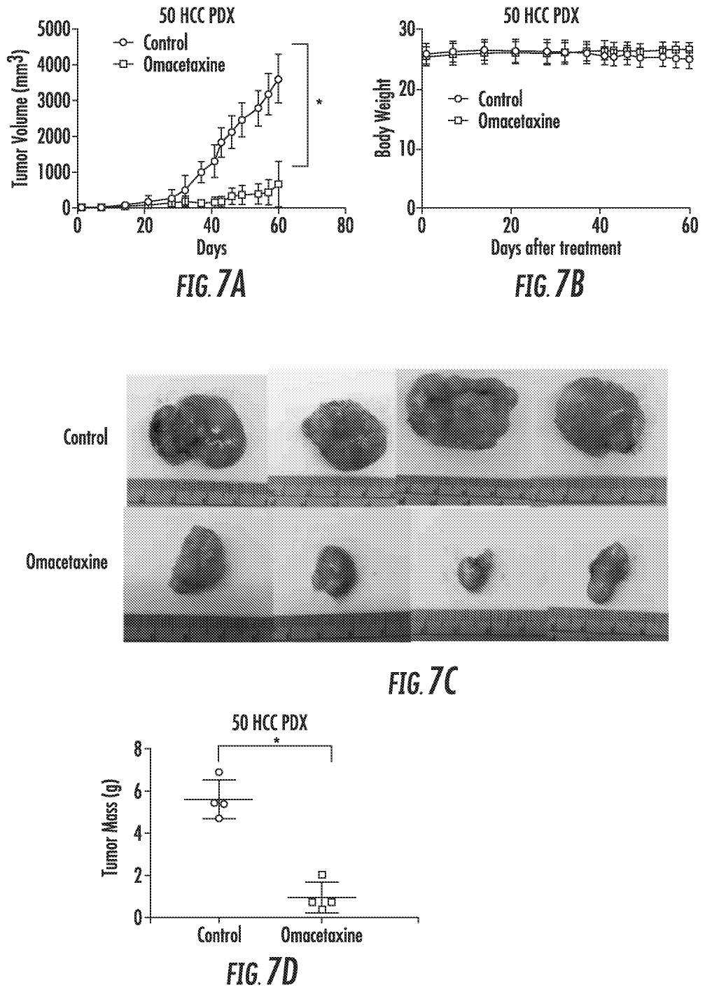

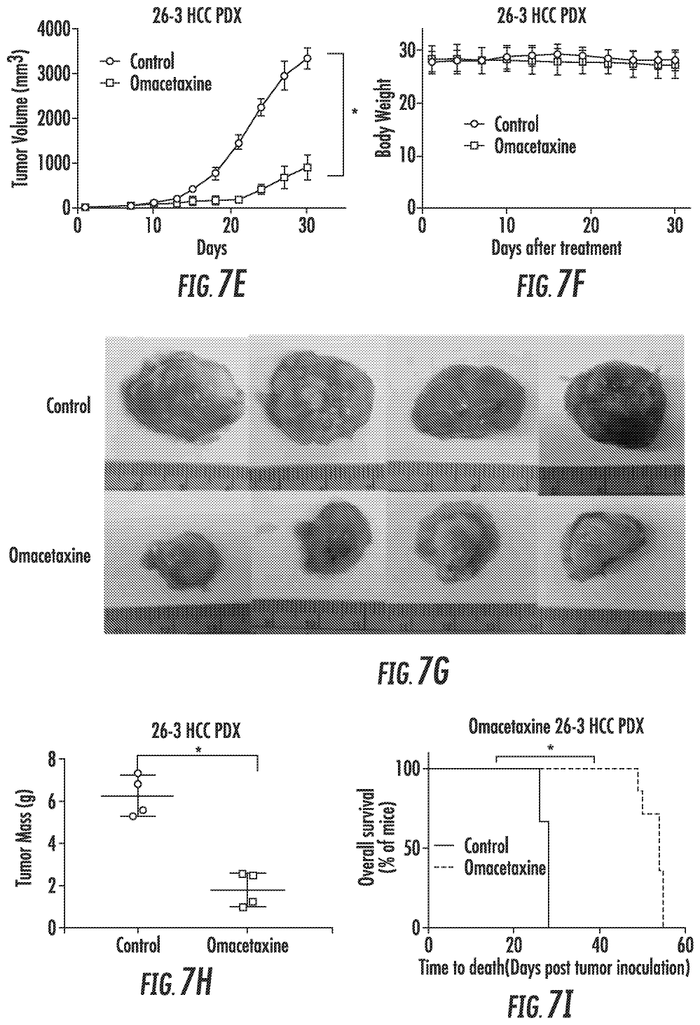

The second model, PDX50, was established by directly implanting pieces of the fresh human HCC tumor into NSG mice. After initial implantation, PDX50 is perpetuated similarly to PDX26-3. Hematoxylin and eosin (H&E) staining of primary human HCCs and matched PDX cancers was performed (data not shown). To investigate if omacetaxine displays effectiveness in vivo, the inventors utilized both PDX26-3 (which is a more aggressive cancer) and PDX50 (a less aggressive cancer). It was found that omacetaxine treatment applied directly into the tumor resulted in halting of tumor growth in both PDX50 as well as PDX26-3 mice (FIGS. 7A and 7E). Of note, there was no difference in the body mass of mice in the treatment vs. the control group (FIGS. 7B and 7F). At the end of the treatment, there was a statistically significant difference in the size of the tumors, as well as in the weight of the tumors, for each of the PDX models used (FIGS. 7C-D and 7G-H). These data show the omacetaxine is effective in vivo in 2 PDX models of human HCC, which predicts its effectiveness in patients. Last, the inventors investigated if there was a survival advantage provided by the treatment with omacetaxine. PDX50 is less aggressive—mice continue to be active, to eat and function normally, in spite of very large tumors. Therefore, PDX50 is not ideal for survival determinations. PDX26-3, however, is more aggressive (mice die due to the cancer burden). A survival experiment in which PDX26-3 was utilized was designed and implemented, and demonstrated a statistically significant increase in survival of approximately 100% (FIG. 7E).

### Example 7

Omacetaxine Inhibits Cancer Proliferation and Increases Apoptosis In Vivo in PDX Models.

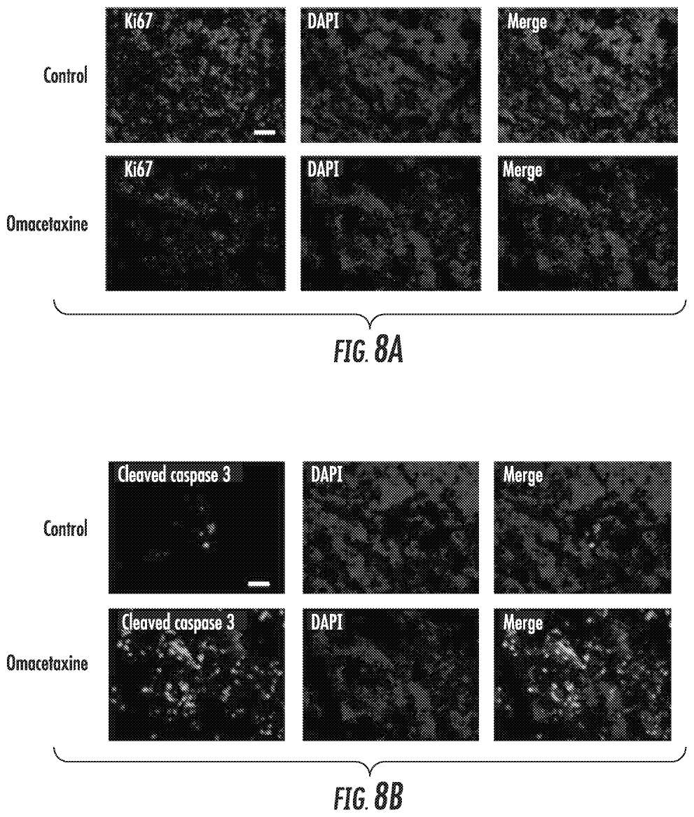

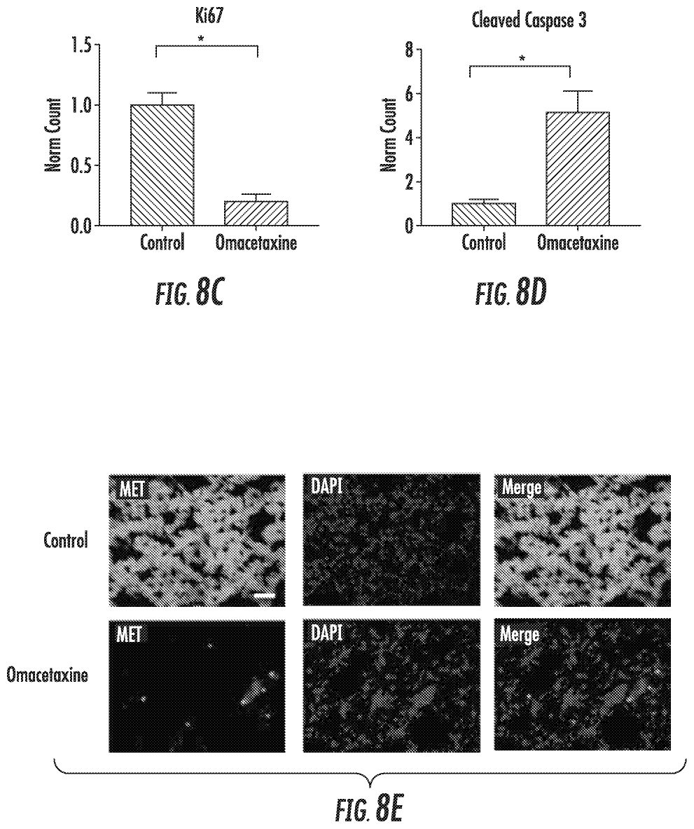

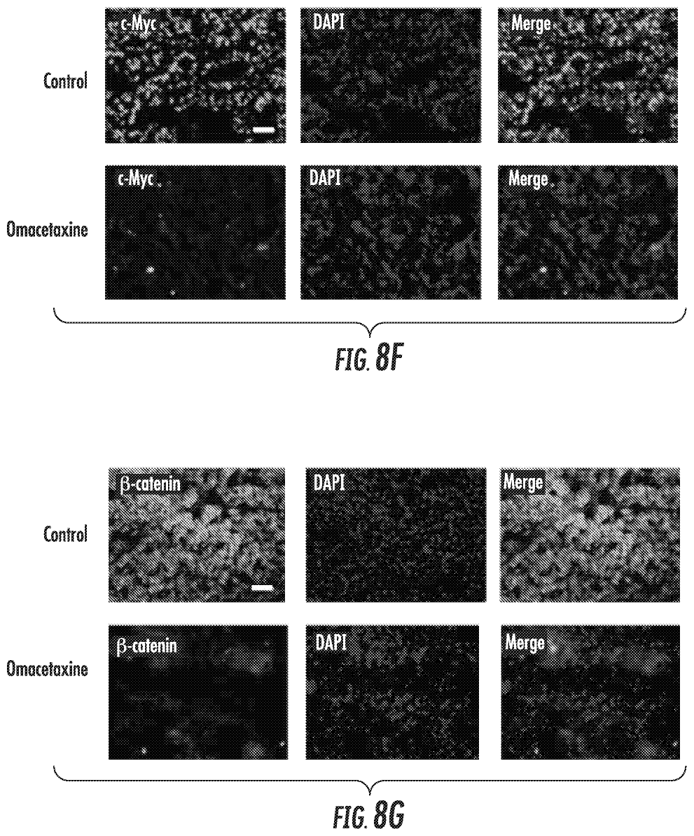

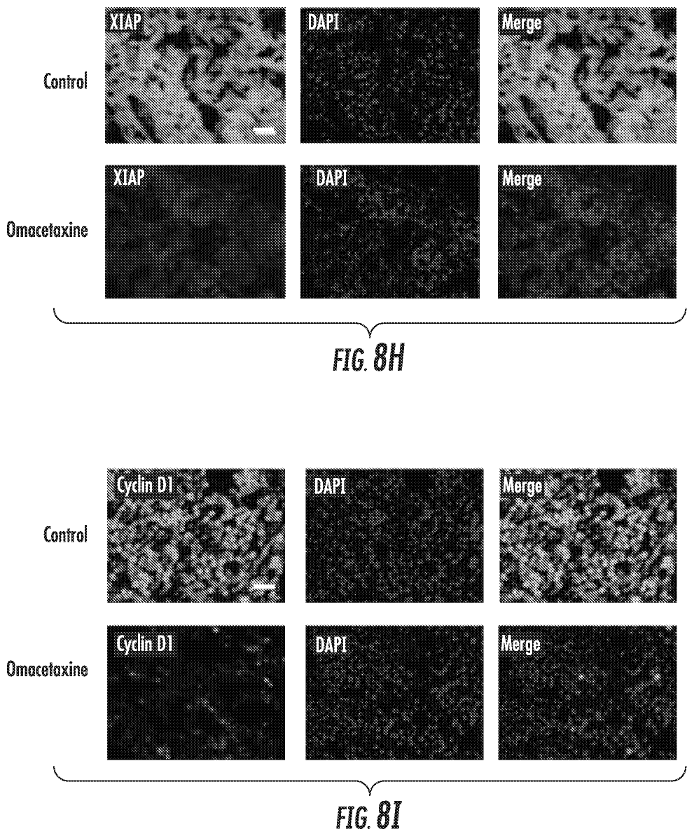

To verify the mechanism of omacetaxine target HCC mechanism in vivo, omacetaxine treated HCC PDX tumors, as well as matched controls, were harvested, and frozen section slides were made. It was found that proliferation was less, as measured by Ki67 protein expression (FIGS. 8A and 8C). Apoptosis was also increased in omacetaxine treated cancers, as quantified by Cleaved Caspase 3 staining (FIG. 8B). The difference in apoptosis as well as proliferation was statistically significant (FIGS. 8C and 8D).

### Example 8

Omacetaxine Downregulates Certain Short Half-Life Oncoproteins in HCC In Vivo.

The inventors demonstrated that omacetaxine induces (1) global downregulation of protein synthesis (FIG. 6), and (2) downregulation of short half-life oncoproteins in vitro. The demonstration of similar findings in vivo would further argue that omacetaxine exerts its effects, at least in part, through inhibition of certain short half-life oncoproteins. Tumor masses from HCC PDX mice treated with omacetaxine and negative control, respectively, were utilized to prepare frozen sections, which were then stained for the c-Myc, MET and beta-catenin. As shown FIGS. 8E-8G, omacetaxine downregulated in vivo the expression of all of these short half-life oncoproteins.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

1. Global Burden of Disease Liver Cancer C, Akinyemiju T, Abera S, et al. The Burden of Primary Liver Cancer and Underlying Etiologies From 1990 to 2015 at the Global, Regional, and National Level: Results From the Global Burden of Disease Study 2015. JAMA Oncol 2017; 3:1683-1691.
2. Llovet J M, Zucman-Rossi J, Pikarsky E, et al. Hepatocellular carcinoma. Nat Rev Dis Primers 2016; 2:16018.
3. Villanueva A. Hepatocellular Carcinoma. N Engl J Med 2019; 380:1450-1462.
4. European Association For The Study Of The L, European Organisation For R, Treatment Of C. EASL-EORTC clinical practice guidelines: management of hepatocellular carcinoma. J Hepatol 2012; 56:908-43.
5. El-Serag H B. Hepatocellular carcinoma. N Engl J Med 2011; 365:1118-27.
6. Abou-Alfa G K, Meyer T, Cheng A L, et al. Cabozantinib in Patients with Advanced and Progressing Hepatocellular Carcinoma. N Engl J Med 2018; 379:54-63.
7. Bruix J, Qin S, Merle P, et al. Regorafenib for patients with hepatocellular carcinoma who progressed on sorafenib treatment (RESORCE): a randomised, double-blind, placebo-controlled, phase 3 trial. Lancet 2017; 389:56-66.
8. Kudo M, Finn R S, Qin S, et al. Lenvatinib versus sorafenib in first-line treatment of patients with unresectable hepatocellular carcinoma: a randomised phase 3 non-inferiority trial. Lancet 2018; 391:1163-1173.
9. Llovet J M, Ricci S, Mazzaferro V, et al. Sorafenib in advanced hepatocellular carcinoma. N Engl J Med 2008; 359:378-90.
10. Broutier L, Mastrogiovanni G, Verstegen M M, et al. Human primary liver cancer-derived organoid cultures for disease modeling and drug screening. Nat Med 2017; 23:1424-1435.
11. Hu H, Gehart H, Artegiani B, et al. Long-Term Expansion of Functional Mouse and Human Hepatocytes as 3D Organoids. Cell 2018; 175:1591-1606 e19.
12. Kopper O, de Witte C J, Lohmussaar K, et al. An organoid platform for ovarian cancer captures intra- and interpatient heterogeneity. Nat Med 2019.
13. Li L, Knutsdottir H, Hui K, et al. Human primary liver cancer organoids reveal intratumor and interpatient drug response heterogeneity. JCI Insight 2019; 4.
14. Sachs N, de Ligt J, Kopper O, et al. A Living Biobank of Breast Cancer Organoids Captures Disease Heterogeneity. Cell 2018; 172:373-386 ell).
15. van de Wetering M, Francies H E, Francis J M, et al. Prospective derivation of a living organoid biobank of colorectal cancer patients. Cell 2015; 161:933-45.
16. Tang R, Faussat A M, Majdak P, et al. Semisynthetic homoharringtonine induces apoptosis via inhibition of protein synthesis and triggers rapid myeloid cell leukemia-1 down-regulation in myeloid leukemia cells. Mol Cancer Ther 2006; 5:723-31.
17. Kuroda J, Kamitsuji Y, Kimura S, et al. Anti-myeloma effect of homoharringtonine with concomitant targeting of the myeloma-promoting molecules, Mcl-1, XIAP, and beta-catenin. Int J Hematol 2008; 87:507-515.
18. Chen Y, Hu Y, Michaels S, et al. Inhibitory effects of omacetaxine on leukemic stem cells and BCR-ABL-induced chronic myeloid leukemia and acute lymphoblastic leukemia in mice. Leukemia 2009; 23:1446-54.
19. Allan E K, Holyoake T L, Craig A R, et al. Omacetaxine may have a role in chronic myeloid leukaemia eradication through downregulation of Mcl-1 and induction of apoptosis in stem/progenitor cells. Leukemia 2011; 25:985-94.
20. Gandhi V, Plunkett W, Cortes J E. Omacetaxine: a protein translation inhibitor for treatment of chronic myelogenous leukemia. Clin Cancer Res 2014; 20:1735-40.
21. Nicolini F E, Khoury H J, Akard L, et al. Omacetaxine mepesuccinate for patients with accelerated phase chronic myeloid leukemia with resistance or intolerance to two or more tyrosine kinase inhibitors. Haematologica 2013; 98:e78-9.
22. Gerdes J, Lemke H, Baisch H, et al. Cell cycle analysis of a cell proliferation-associated human nuclear antigen defined by the monoclonal antibody Ki-67. J Immunol 1984; 133:1710-5.
23. Gerdes J, Li L, Schlueter C, et al. Immunobiochemical and molecular biologic characterization of the cell proliferation-associated nuclear antigen that is defined by monoclonal antibody Ki-67. Am J Pathol 1991; 138:867-73.
24. Luo Y, Ren F, Liu Y, et al. Clinicopathological and prognostic significance of high Ki-67 labeling index in hepatocellular carcinoma patients: a meta-analysis. Int J Clin Exp Med 2015; 8:10235-47.
25. Lam S S, Ho E S, He B L, et al. Homoharringtonine (omacetaxine mepesuccinate) as an adjunct for FLT3-ITD acute myeloid leukemia. Sci Transl Med 2016; 8:359ra129.
26. Chen R, Gandhi V, Plunkett W. A sequential blockade strategy for the design of combination therapies to overcome oncogene addiction in chronic myelogenous leukemia. Cancer Res 2006; 66:10959-66.
27. Thomas J J, Abed M, Heuberger J, et al. RNF4-Dependent Oncogene Activation by

Protein Stabilization. Cell Rep 2016; 16:3388-3400.
28. Aparicio S, Hidalgo M, Kung A L. Examining the utility of patient-derived xenograft mouse models. Nat Rev Cancer 2015; 15:311-6.
29. Zhang X, Claerhout S, Prat A, et al. A renewable tissue resource of phenotypically stable, biologically and ethnically diverse, patient-derived human breast cancer xenograft models. Cancer Res 2013; 73:4885-97.
30. Jemal A, Ward E M, Johnson C J, et al. Annual Report to the Nation on the Status of Cancer, 1975-2014, Featuring Survival. J Natl Cancer Inst 2017; 109.
31. Michelotti G A, Machado M V, Diehl A M. NAFLD, NASH and liver cancer. Nat Rev

Gastroenterol Hepatol 2013; 10:656-65.
32. de Lope C R, Tremosini S, Forner A, et al. Management of HCC. J Hepatol 2012; 56 Suppl 1:S75-87.
33. Kantarjian H M, Talpaz M, Santini V, et al. Homoharringtonine: history, current research, and future direction. Cancer 2001; 92:1591-605.
34. Powell R G, Weisleder D, Smith C R, Jr., et al. Structures of harringtonine, isoharringtonine, and homoharringtonine. Tetrahedron Lett 1970:815-8.
35. Huang M T. Harringtonine, an inhibitor of initiation of protein biosynthesis. Mol

Pharmacol 1975; 11:511-9.
36. Grem J L, Cheson B D, King S A, et al. Cephalotaxine esters: antileukemic advance or therapeutic failure? J Natl Cancer Inst 1988; 80:1095-103.
37. Cortes J, Digumarti R, Parikh P M, et al. Phase 2 study of subcutaneous omacetaxine mepesuccinate for chronic-phase chronic myeloid leukemia patients resistant to or intolerant of tyrosine kinase inhibitors. Am J Hematol 2013; 88:350-4.
38. Cortes J, Lipton J H, Rea D, et al. Phase 2 study of subcutaneous omacetaxine mepesuccinate after T M failure in patients with chronic-phase CML with T3151 mutation. Blood 2012; 120:2573-80.
39. Wang L, Zhao L, Wei G, et al. Homoharringtonine could induce quick protein synthesis of PSMD11 through activating MEK1/ERK1/2 signaling pathway in pancreatic cancer cells. J Cell Biochem 2018; 119:6644-6656.
40. Weng T Y, Wu H F, Li C Y, et al. Homoharringtonine induced immune alteration for an Efficient Anti-tumor Response in Mouse Models of Non-small Cell Lung Adenocarcinoma Expressing Kras Mutation. Sci Rep 2018; 8:8216.
41. Nijenhuis C M, Hellriegel E, Beijnen J H, et al. Pharmacokinetics and excretion of (14)C-omacetaxine in patients with advanced solid tumors. Invest New Drugs 2016; 34:565-74.
42. Chen R, Guo L, Chen Y, et al. Homoharringtonine reduced Mcl-1 expression and induced apoptosis in chronic lymphocytic leukemia. Blood 2011; 117:156-64.

1. Use of an effective amount of a suppressor of protein translation for treating a solid cancer or tumor in a subject.

2. Use of an effective amount of a suppressor of protein translation for treating liver cancer or a liver tumor in a subject.

3. Use of an effective amount of a suppressor of protein translation and at least one additional biologically active agent for treating liver cancer or a liver tumor in a subject.

4. Use of an effective amount of a suppressor of protein translation and at least one additional chemotherapeutic agent for treating liver cancer or a liver tumor in a subject.

5. The use of any of claims 1 to 4, wherein the a suppressor of protein translation is omacetaxine

6. The use of any of claims 1 to 4, wherein the cancer is hepatocellular carcinoma (HCC) and/or cholangiocarcinoma.

7. The use of claim 3, wherein the additional biologically active agent is a tyrosine kinase inhibitor.

8. The use of claim 7, wherein the tyrosine kinase inhibitor is selected from the group consisting of sorafenib, lenvantinib, regorafenib, cabozantinib and ramucirumab.

9. The use of claim 4, wherein the chemotherapeutic agent is selected from the group consisting of alkylating agents, angiogenesis inhibitors, aromatase inhibitors, antimetabolites, anthracyclines, antitumor antibiotics, monoclonal antibodies, platinums, topoisomerase inhibitors, and plant alkaloids.
